(19) United States
(12) Patent Application Publication
(10) Pub. No.: US 2004/0259259 A1
(43) Pub. Date: Dec. 23, 2004

(54) Techniques for analyzing products containing a coloring agent

(76) Inventors: Richard P. Gill, Essex, CT (US); J. Christopher Philips, Charlestown, RI (US)

Correspondence Address: Alan W. Steele, M.D., Ph.D., Wolf, Greenfield & Sacks, P.C., 600 Atlantic Avenue, Boston, MA, 02210, US

(21) Appl. No.: 10/694,637

(22) Filed: Oct. 24, 2003

Related U.S. Application Data
(60) Provisional application No. 60/420,969, filed on Oct. 24, 2002.

(51) Int. Cl. G01N 33/00

(52) U.S. Cl. 436/8; 436/56

(57) ABSTRACT

A method is provided for detecting a tracer in a preparation by precipitating a coloring agent from a sample of the preparation, especially where the coloring agent otherwise interferes with detection of the tracer. A sample of the preparation may be contacted with an effective amount of a precipitating agent to precipitate the coloring agent in the sample, yielding a sample supernatant having a reduced concentration of coloring agent compared to the preparation. Detection of the tracer in the supernatant may produce a result which may be evaluated to assist in quality validation in a process of manufacturing the preparation or authenticating the preparation.

### RELATED APPLICATIONS

This application claims the benefit of co-pending U.S. Provisional Patent Application Ser. No. 60/420,969, filed Oct. 24, 2002.

### FIELD OF THE INVENTION

The invention relates to methods that include detecting a tracer in a sample of a preparation having a coloring agent for the purposes of authentication, process or quality control, or detecting counterfeits.

### BACKGROUND OF THE INVENTION

Distribution of counterfeit substances or production of misformulated material from a licensor/franchisor is a problem for many brand owners; therefore, it may be desirable to test a preparation for authenticity, concentration or quality, during or after production. Process control systems that generally involve the detection, measurement and optionally modulation of a tracer in a preparation, are useful in a number of manufacturing settings.

Process control systems have been previously proposed, such as in PCT Application No. WO 87/06383, European Application No. EP-A-0260829 and commonly assigned U.S. Pat. Nos. 5,249,952, 5,753,511, 6,232,124, 6,458,595, 6,490,030 and 6,512,580, which are hereby incorporated by reference in their respective entireties. Many of these systems involve adding a taggant to a product such that the taggant may be detected at various points during and after production.

Certain products have ingredients or properties that interfere with the detection of a taggant, such as a coloring agent, increased viscosity or a light-diffusing property. To enable these known process control methods to be used to detect a taggant in these products, an increased amount of or a different taggant may be required, thereby possibly affecting the taste, presentation or composition of the product. Clearly, altering the final product in any way is undesirable.

### SUMMARY OF THE INVENTION

The invention presents a novel system for detecting a tracer in a preparation by reducing or decreasing a coloring agent in the preparation that inhibits detection of the tracer, thereby allowing a type and quantity of tracer to be used that does not undesirably or detectably alter the product. The coloring agent may be precipitated out of a sample of the preparation by contacting the sample with an effective amount of precipitating agent, resulting in a sample supernatant that has a reduced concentration of coloring agent compared to the preparation. The tracer may then be detected in the sample supernatant.

According to one aspect, the invention is a method of detecting a tracer in a preparation including a coloring agent and suspected to include a tracer. The method includes the steps of contacting a sample of the preparation with an effective amount of precipitating agent to precipitate the coloring agent in the sample to yield a sample supernatant having a reduced concentration of coloring agent compared to the preparation, and detecting the tracer in the sample supernatant.

According to another aspect, the invention is a method for verifying the authenticity of a preparation. This method includes the steps of contacting an authentic sample of an authentic preparation, wherein the authentic preparation includes a tracer in an authentic amount and a coloring agent, with an authentic effective amount of a precipitating agent to precipitate the coloring agent in the authentic sample to yield an authentic sample supernatant having an authentic reduced concentration of coloring agent compared to the authentic preparation; detecting the tracer in the authentic sample supernatant; contacting a test sample of a test preparation, wherein the test preparation includes the tracer in a test amount and the coloring agent, with a test effective amount of the precipitating agent to precipitate the coloring agent in the test sample to yield a test sample supernatant having a test reduced concentration of coloring agent compared to the test preparation; detecting the tracer in the test sample supernatant; and determining the test preparation is authentic when the tracer in the test sample supernatant is within a predetermined range of the tracer in the authentic sample supernatant.

According to a further aspect, the invention is a method for quality validation of a preparation. The method includes the steps of contacting a standard sample of a standard preparation, wherein the standard preparation includes a tracer in a standard amount and a coloring agent, with a standard effective amount of a precipitating agent to precipitate the coloring agent in the standard sample to yield a standard sample supernatant having a standard reduced concentration of coloring agent compared to the standard preparation; and detecting the tracer in the standard sample supernatant to produce a standard result; contacting a test sample of a test preparation, wherein the test preparation includes the tracer in a test amount and the coloring agent, with a test effective amount of the precipitating agent to precipitate the coloring agent in the test sample to yield a test sample supernatant having a test reduced concentration of coloring agent compared to the test preparation; detecting the tracer in the test sample supernatant to produce a test result; and determining the test preparation is satisfactory when the test result is within a predetermined range of the standard result.

According to yet another aspect, the invention is a method of analyzing a product, the product including a coloring agent and suspected of comprising a tracer. The method includes the steps of contacting at least a sample of the product with an effective amount of a precipitating agent to precipitate the coloring agent in the sample to yield a supernatant of the product having a reduced concentration of coloring agent compared to the sample of the product; and detecting the tracer in the supernatant of the product.

The preparation may be a liquid consumable product, such as a cola beverage and a concentrate of a cola beverage. The coloring agent may be a caramel coloring agent, such as a Type 1 caramel color and may otherwise interfere with detecting the tracer. The reduced concentration of coloring agent may be between 0 and 10 percent, 0 and 1 percent or 0 and 0.1 percent compared to the liquid consumable product.

In one embodiment the precipitating agent specifically precipitates the coloring agent and not the tracer. The precipitating agent may comprise a Type 2 caramel color, a Type 3 caramel color, a cationic or quaternary surfactant or a polymer thereof, a quaternary ammonium bromide, such as myristyltrimethylammonium bromide (MYTAB), a quaternary ammonium chloride, a copolymer of quaternary acrylate salt and acylamide, a copolymer sodium acrylate and an acrylamide, a polyacrylamide, or a polyamine. Additionally, the pH of the sample may be adjusted prior to contacting the sample with the precipitating agent.

The tracer may be directly or indirectly detectable and may comprise a food dye or a light-sensitive compound. In one embodiment the tracer is FD&C Blue #1. The tracer may be present in 100-2000 parts per billion (ppb) of the preparation. The step of detecting the tracer may comprise detecting an optical property of the tracer. The optical property may be an absorbance or emittance at a predetermined wavelength. Detecting the tracer may include detecting the existence of the tracer or detecting the concentration of the tracer.

The at least a sample of the product may be the entire product. The preparation or product may be a liquid preparation.

Further features and advantages of the present invention as well as the methods of various embodiments of the present invention are described in detail below.

### DETAILED DESCRIPTION OF THE INVENTION

The invention broadly relates to detecting a tracer in a preparation. More specifically, the invention is directed to a method of removing a coloring agent from a sample of the preparation, enabling a user to detect the tracer. The parameters that can be measured and subsequently adjusted include presence and concentration of particular tracers such as the ingredients (e.g., substrates), by-products, catalysts, and end-products of a process or an additional substance not required in producing the preparation. Thus, the methods provided herein generally relate to the removal of a coloring agent from a sample such that one or more tracers can be detected and quantified. Detection and quantitation of tracers from a sample having a coloring agent removed or reduced in concentration, can be similarly applied to product verification (e.g., product authentication) strategies.

A sample of a preparation that contains a tracer and a coloring agent may be contacted with a precipitating agent in an effective amount to precipitate out at least a portion of the coloring agent. A sample supernatant having a concentration of coloring agent less than that of the preparation may result. The tracer may be detected in the sample supernatant having a reduced concentration of coloring agent. One advantage of this embodiment is that a lesser amount of tracer may be used; for the tracer to be detectable, a liquid having a greater or normal concentration of coloring agent requires more tracer than a supernatant with a reduced concentration of coloring agent. In fact, a feature of the invention is the ability to detect a surprisingly small concentration of tracer (parts per billion) in a preparation having a coloring agent that otherwise interferes with detection of the tracer (see Examples 1-5 and 7-8).

This method may be performed on a sample with a standard or authentic concentration of tracer, such that a known result of the tracer detection will be expected. This known result can be compared to a result from an unknown or test sample, providing information on detection and/or concentration of the tracer.

Preparation

The samples analyzed according to the methods of the invention are derived (i.e., harvested or extracted) from a larger preparation referred to herein as the “preparation.” Rather than test the preparation directly, the sample is harvested and tested, thereby reducing the manipulation of the preparation. The preparation may be a bulk preparation. In one embodiment the preparation is a liquid preparation. It should be appreciated that the preparation need not be a liquid and may have properties like that of a solid, gas or plasma.

Any Product

The preparation may include any chemical-type product to be tested, such as consumable (i.e., food and beverage) products, pharmaceuticals (e.g., chemotherapeutics or headache medication), cosmetics (e.g., hairspray or liquid concealer), industrial products (e.g., paint, glue, fuels such as fuel oil), and the like, as the present invention is not limited in this respect. Consumable products include foodstuffs, syrups, concentrates, alcoholic beverages, non-alcoholic beverages such as colas, and the like.

Tracer

The preparation may contain a tracer to be detected. As used herein, a “tracer” is an element, a compound, or in some instances a family of compounds sharing a common feature, that may be detected in the preparation. The tracer may include, but is not limited to, protein, peptide (e.g., a protein fragment), glycoprotein, carbohydrate, polysaccharide, receptor, ions (such as sodium, fluoride, calcium, potassium), hormone, growth factor, antibody, antigen, a pathogen such as a bacterium, a virus, or a parasite (or a fragment or particle thereof), enzyme, substrate, cofactor, inhibitor, metabolite, transition state analog, drug, dye, nutrient, light-sensitive compound such as light-emissive or light-absorptive compounds, fluorescent materials, such as quinine or many other aromatic or phenolic materials and the like.

In embodiments in which the sample is a food or beverage, the tracer may be but is not limited to vitamin, food additive, coloring or food dye, such as FD&C Blue, Red or Yellow available from Prime Ingredients Incorporated located in Saddle Brook, N.J., preservative, flavoring, sweetener, anti-oxidant, and the like. Specific examples of tracers that can be determined will depend upon the sample being tested yet include sugar, aspartame, caffeine, vanilla, juice (e.g., lime juice), phosphoric acid, alcohol, oil essences, seasonings such as cinnamon, nutmeg, and the like, although detection of other tracers is contemplated.

Tracers may be ingredients (e.g., substrates), additives, final components (i.e., end-products), or by-products of manufacture of the preparation. For example, if a cola is produced by combining a syrup with carbonated water, then ingredients or end-products, or some combination thereof, can be measured in order to assess characteristics (e.g., authenticity of cola, concentration of the syrup, etc.) of the preparation and/or its ingredients during the manufacturing process. If the preparation does not inherently contain or produce a desirable tracer, a tracer may be added to the preparation.

The tracer may be present in any amount in the preparation as long as the tracer, when present, may be detected using a method of the invention. In one embodiment, the tracer is present in 100-2000 ppb of the preparation. In certain embodiments, such as when the preparation is not authentic, the tracer may not present in any detectable quantity in the sample and/or in the preparation.

It should be appreciated that the tracer should not be specifically precipitated by the precipitating agent, as will be described further below.

Unknown Tracer

In some embodiments of the invention, the tracers being detected are known and thus the detection result is of known tracers. The invention however is not so limited, as it is possible to determine a detection result without knowledge of the tracers being detected. This is particularly true for authentication methods where a profile of a test sample is being compared to a profile of an authentic sample.

Coloring Agent

The preparation may also contain a coloring agent, at least a portion of which will be precipitated to enable detection of the tracer. As used herein, a “coloring agent” is any colored or non-clear property of the preparation that may interfere with the detection of the tracer. For example, the coloring agent may be any feature that inhibits the passage of light through the preparation, such as color, viscosity, or other light-diffusing properties.

In one embodiment the coloring agent is caramel color. Caramel color is a complex commercial product manufactured by cooking sugars with various catalysts to achieve a desired color and related properties. Caramel color is commonly found in products including soft drinks, such as colas, root beers, cream sodas, orange sodas, and ginger ales, and alcoholic beverages, notably tequila and scotch.

Caramel color may serve the function of delivering color to the preparation, protecting flavors from light deterioration and emulsifying the preparation, possibly reducing the need for gums. Caramel color may be in liquid or solid form and may be found in beverages, such as soft drinks or syrups thereof, powdered soft drinks, fruit drink mix, beer and ale, malt drinks, soy drinks, iced tea, iced tea mix, iced coffee, instant coffee flavor enhancer, cappuccino mix, dairy or yogurt, instant chocolate milk, hot cocoa mix, nutritional (protein) drink mix, wine coolers, liquors; sweets, such as cocoa extender, artificial vanilla extract, dark breads and cakes, bread mix, frozen/refrigerated dough, croutons, stuffing, muffins, bagels, muffin mix, snack cakes, chocolate cake mix, cookies, biscuits, crackers, ice cream sandwich wafers, ice cream cones, striped wafer sticks, ice cream frozen novelties, instant pudding, pie fillings, syrups, toppings, fruit fillings, spreads, cinnamon rolls, sandwich cookie fillings, frostings, icing, brown sugar, black licorice, gummy candy, jelly beans, (boiled) hard candy; cereals and snacks, such as breakfast cereals, instant oatmeal/porridge, nutrition/energy bars, rice cakes, caramel popcorn, yeast extract spread, extruded snacks, snack dusting/seasoning; sauces or seasonings, such as soy sauce, fish/oyster sauce, cooking wine, Worcestershire sauce, BBQ/steak sauce, baked bean sauce, mustard, salad dressings and dips, gravies, frozen meals, soups and bases, instant noodle seasoning, bouillon beef cubes, chicken cubes, turkey cubes, fish cubes, spice blends/seasonings, seasoned rice mix, stir-fry mix, breadings and coatings, fish batter, marinades and meat rubs, textured vegetable protein, hydrolyzed vegetable protein, savory flavors; meat products, such as canned meat, meat pie fillings, cooked meatballs, sausage casings, cooked poultry and fish, vegetarian burgers, meat analogues; and pet food, such as batch consistency, extruded/dry, canned/wet, semi moist, gravy, treats/snacks, synthetic color replacer, vitamin supplements. Examples of the foregoing may be found at http://www.caramel.com, “D. D. Williamson: World Leader in Caramel Color”.

In another embodiment, the coloring agent may be any substance approved for use in human food, drugs, cosmetics or medical devices or any substance that is generally recognized as safe (GRAS). Examples of substances approved for use in human food include annatto extract, dehydrated beets (beet powder), canthaxanthin, caramel, β-apo-8′-carotenal, β-carotene, cochineal extract, carmine, sodium copper chlorophyllin, toasted partially defatted cooked cottonseed flour, ferrous gluconate, ferrous lactate, grape color extract, grape skin extract (enocianina), synthetic iron oxide, fruit juice, vegetable juice, carrot oil, paprika, paprika oleoresin, riboflavin, saffron, titanium dioxide, turmeric, turmeric oleoresin, FD&C Blue No.1, FD&C Blue No.2, FD&C Green No. 3, Orange B, Citrus Red No. 2, FD&C Red No.3, FD&C Red No. 40, FD&C Yellow No. 5 and FD&C Yellow No. 6.

Examples of substances approved for use in drugs include alumina (dried aluminum hydroxide), annatto extract, calcium carbonate, canthaxanthin, caramel, β-carotene, cochineal extract, carmine, potassium sodium copper chlorophyllin (chlorophyllin-copper complex), dihydroxyacetone, bismuth oxychloride, synthetic iron oxide, ferric ammonium ferrocyanide, ferric ferrocyanide, chromium hydroxide green, chromium oxide greens, guanine, pyrophillite, mica, talc, titanium dioxide, aluminum powder, bronze powder, copper powder, zinc oxide, FD&C Blue No. 1, FD&C Blue No. 2, D&C Blue No. 4, FD&C Green No. 3, D&C Green No. 5, D&C Green No. 6, D&C Green No. 8, D&C Orange No. 4, D&C Orange No. 5, D&C Orange No. 10, D&C Orange No. 11, FD&C Red No. 3, FD&C Red No. 4, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, D&C Red No. 39, FD&C Red No. 40, D&C Violet No. 2, FD&C Yellow No. 5, FD&C Yellow No. 6, D&C Yellow No. 7, Ext. D&C Yellow No. 7, D&C Yellow No. 8, D&C Yellow No. 10 and D&C Yellow No. 11.

Examples of substances approved for use in cosmetics include annatto, caramel, carmine, β-carotene, bismuth citrate, disodium EDTA-copper, potassium sodium copper chlorophyllin (chlorophyllin copper complex), dihydroxyacetone, bismuth oxychloride, guaiazulene, henna, iron oxides, ferric ammonium ferrocyanide, ferric ferrocyanide, chromium hydroxide green, chromium oxide greens, guanine, lead acetate, pyrophyllite, mica, silver, titanium dioxide, aluminum powder, bronze powder, copper powder, ultramarines, manganese violet, zinc oxide, luminescent zinc sulfide, FD&C Blue No. 1, D&C Blue No. 4, D&C Brown No. 1, FD&C Green No. 3, D&C Green No. 5, D&C Green No. 6, D&C Green No. 8, D&C Orange No. 4, D&C Orange No. 5, D&C Orange No. 10, D&C Orange No. 11, FD&C Red No. 4, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, FD&C Red No. 40, D&C Violet No. 2, Ext. D&C Violet No. 2, FD&C Yellow No. 5, FD&C Yellow No. 6, D&C Yellow No. 7, Ext. D&C Yellow No. 7, D&C Yellow No. 8, D&C Yellow No. 10 and D&C Yellow No. 11.

Examples of substances approved for use in medical devices include chromium-cobalt-aluminum oxide, ferric ammonium citrate, pyrogallol: C.I. oxidation base 32, logwood extract; C.I. natural black 1, 1,4-bis[(2-hydroxy-ethyl)amino]-9,10-anthracenedione bis(2-propenoic)ester copolymers, 1,4-bis[(2-methylphenyl)amino]-9,10-anthracenedione, 1,4-bis[4-(2-methacryloxyethyl)phenylamino]anthraquinone copolymers, carbazole violet, chlorophyllin-copper complex, oil soluble, chromium-cobalt-aluminum oxide, chromium oxide greens, C.I. vat orange 1, 2-[(2,5-diethoxy-4-[(4-methylphenyl)thiol]phenyl)azo]-1,3,5-benzenetriol, C.I. vat brown 1: 16,23-dihydrodinaphtho[2,3-a:2′,3′-i]naphth[2′,3′:6,7]indolo[2,3-c]carbazole-5,10,15,17,22,24-hexone, C.I. vat yellow 3: N,N′-(9,10-dihydro-9,10-dioxo-1,5-anthracenediyl)bisbenzamide, C.I. vat blue 6: 7,16-dichloro-6,15-dihydro-5,9,14,18-anthrazinetetrone, C.I. vat green 1: 16,17-dimethoxydinaphtho(1,2,3-cd:3′,2′,1′-1 m)perylene-5,10-dione, poly(hydroxyethyl methacrylate)-dye copolymers and one or more of C.I. reactive black 5, C.I. reactive blue 21, C.I. reactive orange 78, C.I. reactive yellow 15, C.I. reactive blue 19, C.I. reactive blue 4, C.I. reactive red 11, C.I. reactive yellow 86, C.I. reactive blue 163 or C.I. reactive red 180, C.I. solvent yellow 18: 4-[(2,4-dimethylphenyl)azo]-2,4-dihydro-5-methyl-2-phenyl-3H-pyrazol-3-one, C.I. vat orange 5: 6-Ethoxy-2-(6-ethoxy-3-oxobenzo(b)thien-2(3H)-ylidene)benzo(b)thiophen-3(2H)-one, phthalocyanine green, iron oxides, titanium dioxide, vinyl alcohol/methyl methacrylate-dye reaction products and one or more of: C.I. reactive red 180, C.I. reactive black 5, C.I. reactive orange 78, C.I. reactive yellow 15, C.I. reactive blue 19 or C.I. reactive blue 21, D&C Blue No. 9, D&C Green No. 5, (Phthalocyaninato(2-)) copper, FD&C Blue No. 2, D&C Blue No. 6, D&C Green No. 6, D&C Red No. 17, D&C Violet No. 2 and D&C Yellow No. 10.

The coloring agent may be ionically charged. For example, in one embodiment caramel color constituents are electron-rich, making the caramel color anionically or partially anionically charged. The coloring agent may be anionic, cationic, amphoteric, monovalent, bivalent, trivalent, quaternary, or have any positive or negative charge. Additionally, polar compounds may be used.

Sample

To preserve the integrity of at least a portion of the preparation, a sample of the preparation may be removed, from which the coloring agent may be precipitated. As used herein, a “sample” is any portion of the preparation that contains or is suspected of containing a tracer, the presence and concentration of which can be detected. Generally, a sample is an aliquot of a larger amount of fluid or solid that is being tested (i.e., the preparation). The sample (and the larger fluid or solid source from which it derives) can contain more than one tracer, and it is possible using the methods of the invention to test for the presence and measure the concentration of each tracer therein contained.

It should be appreciated that the sample may be the entire preparation. In this embodiment, a precipitating agent may be added to the entire preparation and the tracer in the resulting supematant may be detected.

It is to be appreciated that the sample to be tested may be a liquid, a solid such as a dry powder material, a plasma or a gas. Although the methods described herein can be applied to both wet and dry forms of a sample, in some embodiments, it may be preferable to solubilize a dry form prior to detection. In these situations, the signals produced by the sample should be compared to the signals produced from the solubilizing liquid in order to identify sample-specific signals. It is also envisioned that samples can be either diluted or concentrated prior to detection. Use of detection devices with a broad spectrum of capabilities for detecting a particular tracer can in some instance preclude the need for dilution or concentration of the sample.

Precipitating Agent

To remove the coloring agent from the sample, a precipitating agent may be employed. As used herein, a “precipitating agent” is any substance that will precipitate the coloring agent out of the preparation. The precipitating agent may be introduced into the sample in the form of a liquid, a solid, or a solid attached to a substrate.

Without meaning to be bound to any theory or mechanism, the precipitating agent may have an opposite ionic charge compared to the coloring agent. Opposing charges allow the precipitating agent to bind to the coloring agent and remove it from the solution. For example, if the coloring agent is anionic, the precipitating agent may be cationic. It should be appreciated that the precipitating agent may work in anyway to remove the coloring agent from solution.

In certain embodiments, the precipitating agent may be a cationic surfactant or a polymer thereof, a quaternary surfactant or a polymer thereof, a quaternary ammonium bromide or a polymer thereof, a quaternary ammonium chloride or a polymer thereof, a copolymer of a quaternary acrylate salt and acylamide, a copolymer of sodium acrylate and acrylamide, a polyacrylamide, a polyamine, or other amine, ammonium or sulfonium functionalities.

Examples of a quaternary ammonium bromide include, but are not limited to, n-hexadecyltrimethylammonium bromide and tetramethylammonium bromide, available from Alfa Aesar located in Ward Hill, Mass., myristyltrimethylammonium bromide (MYTAB), available from Aldrich located in Milwaukee, Wis., CTAB (cetyltrimethylammonium bromide), available from FeF Chemicals A/S located at K\acute{ø}benhavnsvej 216, 4600 K\acute{ø}ge, Denmark, and hexadecyltrimethylammonium bromide, available from Fluka Chemical Corporation located in Milwaukee, Wis.

Examples of a quaternary ammonium chloride include, but are not limited to, MKC (myristalkonium chloride), available from FeF Chemicals A/S, Empigen BCM 83 (Alkyl dimethyl benzyl ammonium chloride), Empigen HBC 40 (Alkyl dimethyl hydroxyethyl ammonium chloride) and Emipgen BAC50, available from Huntsman located in Oldbury, West Midlands, UK, and n-hexadecyltrimethylammonium chloride, available from Alfa Aesar.

Examples of a copolymer of a quaternary acrylate salt and acylamide include, but are not limited to, Zetag 7503, Zetag 7553, Zetag 7557, Zetag 7563, Zetag 7565, Zetag 7587, Zetag 7634, Zetag 7635, Zetag 7645, Zetag 7651, Zetag 7664, Zetag 7689, Zetag 7692, Zetag 8660, and Magnafloc LT22S, all available from Ciba Specialty Chemicals Corporation located in Suffolk, Va.

Examples of a copolymer of sodium acrylate and acrylamide include, but are not limited to, Magnafloc LT25, Magnafloc LT26, Magnafloc LT27, Magnafloc 10, Magnafloc 24, Magnafloc 155, Magnafloc 336, Magnafloc 338, Magnafloc 340, Magnafloc 342, Magnafloc 919, Magnafloc 1011, and Alclar 662, all available from Ciba Specialty Chemicals Corporation.

Examples of a polyacrylamide include, but are not limited to, Magnafloc 333, Magnafloc 351, Magnafloc 358, Magnafloc E30, Magnafloc LT20 and Magnafloc LT7922, all available from Ciba Specialty Chemicals Corporation.

Examples of a polyamine include, but are not limited to, Magnafloc LT7990 and Magnafloc LT7991, both available from Ciba Specialty Chemicals Corporation.

Additionally, the precipitating agent may include, but is not limited to, an acrylate polymer, such as Magnafloc 611, available from Ciba Specialty Chemicals Corporation, an anionic polyacrylamide emulsion, such as Magnafloc E32 and Magnafloc E38, both available from Ciba Specialty Chemicals Corporation, and an organic cationic polyelectrolyte, such as Magnafloc LT7992 and Magnafloc LT7995, both available from Ciba Specialty Chemicals Corporation.

In one embodiment wherein a Type 1 caramel color (also known as “acid-proof” or “beverage type”) is employed in the preparation as the coloring agent, a Type 2 caramel color (also known as “brewer's color”) or a Type 3 caramel color (also known as “baker's color”) may be used as a precipitating agent. Type 1 has electronegative colloidal characteristics and will be precipitated by an agent having an electropositive character, such as Type 2 or Type 3. Correspondingly, if Type 2 or Type 3 is used as the coloring agent, Type 1 may act as a precipitating agent.

Water-soluble alcohols, such as methyl, ethyl, propyl, isopropyl or butyl alcohol, ethers, such as methyl ether, ethyl ether, isopropyl ether, lower aliphatic glycol ethers, such as dioxane, ketones and similar hydrophilic liquids, such as are disclosed in U.S. Pat. Nos. 2,533,211, 2,637,655 and 2,902,393, for example, may also be used to precipitate the coloring agent.

To facilitate the precipitation process, various conditions may be adjusted. These conditions include, but are not limited to pH, temperature, pressure, exposure to light and aeration. The precipitating agent may be the factor that causes the change of a particular condition. For example, if an increase in temperature causes the coloring agent to precipitate, the precipitating agent may be a heater, or components that when mixed produce an exothermic reaction, thereby increasing the temperature. In one embodiment wherein the coloring agent is Type 1 caramel color, a strong acid, such as hydrochloric acid, may be the precipitating agent, by lowering the pH of the sample.

It should be further appreciated that more than one precipitating agent may be used to precipitate the coloring agent. In one embodiment, two precipitating agents are used, such as MYTAB and MKC. In another embodiment a condition or factor may be adjusted, added or removed to enable the precipitating agent to more effectively precipitate the coloring agent. For example, in one embodiment wherein the coloring agent is a Type 1 caramel color, a strong base such as potassium hydroxide, available from Sigma Chemical Corporation in St. Louis, Mo., and MYTAB may be added. The potassium hydroxide may raise the pH, making the Type 1 caramel color more anionic, thereby enabling the MYTAB to more effectively precipitate the Type 1 caramel color.

Although specific examples are listed, the present invention is not limited in this respect and any other suitable precipitating agent may be used.

Effective Amount

The precipitating agent should be introduced in an effective amount to precipitate at least a portion of the coloring agent. As used herein, “an effective amount” is an amount of precipitating agent that will precipitate at least a portion of the coloring agent, yielding a sample supernatant having a lesser concentration of coloring agent than the concentration of the preparation or the sample before the precipitating agent was added. In one embodiment the effective amount is an excess amount relative to the coloring agent to be precipitated from the sample, such that essentially all of the coloring agent is precipitated from the sample.

Sample Supernatant

Once the precipitating agent is contacted with the sample, the sample includes two portions: a precipitated portion and a remaining liquid portion, termed the sample supernatant. As used herein, “sample supernatant” refers to the portion of the sample that is not the precipitated portion. In most cases the sample supernatant will be a liquid solution. The precipitated portion may be physically contained in or near the sample supernatant, but the precipitated portion should not be considered part of the sample supernatant. In one embodiment, the precipitated portion is removed from the supernatant. This can be accomplished, for example, by one or more decantation, filtration or centrifugation steps.

Reduced Concentration

The sample supernatant will have a reduced concentration of coloring agent as compared to the concentration of coloring agent in the preparation. As used herein, “reduced concentration” is the concentration of coloring agent in the sample supernatant and is less than the concentration of coloring agent in the preparation. For example, if the liquid concentration has a coloring agent concentration of 1000 ppm and the sample supernatant has a coloring agent concentration of 10 ppm; the reduced concentration is the 10 ppm of coloring agent in the sample supernatant. It should be appreciated that concentrations may be expressed in any way, such as concentration, percent, fraction, etc., as the present invention is not intended to be limiting in this respect.

In one embodiment, the sample supernatant will have a reduced concentration of coloring agent as compared to the concentration of coloring agent in the sample; therefore, the reduced concentration of the sample supernatant is less than the concentration of coloring agent in the sample. In various embodiments the reduced concentration is 0 to 10 percent, 0 to 1 percent or 0 to 0.1 percent. It should be appreciated that the reduced concentration may be greater than 10 percent.

Detecting the Tracer

The reduced concentration of coloring agent in the sample supernatant may allow the tracer to be more easily detected. The mere presence or the concentration of the tracer may be detected in any manner. The tracer may be directly or indirectly detected. As used herein, “direct detection” is a method by which the tracer itself is detected. In contrast and as used herein, “indirect detection” incorporates the use of another substance, which either binds to or is affected by the tracer, wherein the substance or affected state thereof is detected.

Direct detection may be accomplished by any suitable method as the present invention is not limited in this respect. In one embodiment, direct detection includes measuring the absorbance or reflection of the tracer in the sample supernatant in response to irradiation at a certain wavelength. In one embodiment, a sample supernatant may be inserted into a spectrophotometer, wherein the sample supernatant may irradiated with a light of a certain wavelength. The sample supernatant will absorb a certain fraction of the light and will transmit a certain percentage. These results can be used to determine the presence of and/or amount of tracer in the sample supernatant.

In one embodiment, the tracer may be directly detected using methods described in commonly assigned U.S. Pat. No. 6,232,124, which is hereby incorporated by reference in its entirety. The tracer itself may be a light-sensitive compound, such that when the sample supernatant is irradiated with light of an appropriate wavelength, the tracer and sample supernatant emit light at a wavelength in response to the irradiating wavelength. The test result may then be compared with an authentic or standard result.

Indirect detection may include the detection of an analyte that interacts with the tracer using any suitable technique as the present invention is not limited in this respect. As used herein, “analyte” may be any detectable substance that will interact with the tracer. For example, the analyte may include, but is not limited to, protein, peptide (e.g., a protein fragment), glycoprotein, carbohydrate, polysaccharide, receptor, ions (such as sodium, fluoride, calcium, potassium), hormone, growth factor, antibody, antigen, a pathogen such as a bacterium, a virus, or a parasite (or a fragment or particle thereof), enzyme, substrate, cofactor, inhibitor, metabolite, transition state analog, drug, dye, nutrient, light-sensitive compound, fluorescent materials, such as quinine or many other aromatic or phenolic materials and the like.

In one embodiment, the tracer is indirectly detected using methods described in commonly assigned U.S. Pat. No. 6,512,580, which is hereby incorporated by reference in its entirety. The sample supernatant may be exposed to a microplate having micropores which contain an analyte that is a light-emissive compound. The sample supernatant and the tracer therein may be allowed to interact with the light-emissive compounds. The tracer may bind to some of the light-emissive compounds. The microplate may then be irradiated with a predetermined wavelength of light. In response, the components on the surface of the microplate, which may include the light-emissive compounds, analytes and/or tracers, will emit a wavelength in response to the irradiating light, providing a test result. This test result may be compared to an authentic or standard result and any difference between the two will signify a difference between the test preparation and the authentic or standard preparation.

In addition or alternatively, the tracer may be detected using the system of commonly assigned U.S. Pat. No. 6,490,030, which is hereby incorporated by reference in its entirety. The system may include a chip having analytes, such as light-emissive compounds, attached to its surface and an authentication device having a light source for irradiating the sample supernatant, an optical detector for detecting an emitted wavelength of light, and a controller for comparing a test result to an authentic or standard result. The tracers in the sample supernatant may interact with the light-emissive compounds to produce an emitted wavelength in response to irradiation from the light source.

Method

In one aspect of the invention, a test sample from a test preparation can be compared with an authentic or standard sample from an authentic or standard preparation. As used herein, “authentic” or “standard” preparation may be any preparation other than the test preparation. The same method of detecting the tracer in a test preparation may be followed on an authentic or standard preparation to provide a basis of comparison for the test sample. It should be appreciated that the authentic or standard sample need not have known characteristics or qualities, other than the results of tracer detection in its sample supernatant following contact with an effective amount of precipitating agent.

In one embodiment the conditions and factors of the authentic or standard precipitation and detection are identical to the conditions and factors of the test precipitation and detection. In this embodiment, both samples may contain the same amount of tracer, the same amount of coloring agent, require the same effective amount of precipitating agent, and yield a sample supernatant having the same reduced concentration of coloring agent.

In another embodiment, the conditions and factors of the authentic or standard precipitation and detection may differ from the conditions and factors of the test precipitation and detection. According to one expression of this embodiment, the authentic or standard sample may contain a different amount of coloring agent than the test sample, possibly requiring different effective amounts of precipitating agent. It should be appreciated that in this expression, the same effective amount of precipitating agent may be used for the test sample as for the authentic or standard sample, provided the effective amount of precipitating agent will yield a sample supernatant having a reduced concentration of coloring agent.

According to another expression of this embodiment, the authentic or standard sample may require a different effective amount of precipitating agent than the test sample, possibly resulting in different reduced concentrations of coloring agent. It should be appreciated that this expression may result in the test sample supernatant having the same reduced concentration of coloring agent as the authentic or standard sample supernatant.

According to a further expression of this embodiment, the authentic or standard sample supernatant may have a different reduced concentration of coloring agent than the test sample supernatant. According to yet another expression of this embodiment, the authentic or standard sample may contain a different amount of tracer than the test sample supernatant. It should be appreciated that any combination of the foregoing or other expressions may be embodied in this embodiment of the method, as the present invention is not intended to be limiting in this respect.

Predetermined Range

To determine that a test preparation is satisfactory or authentic, the outcome of the detection of the tracer in the test sample supernatant will be within a predetermined range of the outcome of the detection of the tracer in the authentic or standard sample supernatant. This predetermined range may vary depending upon the detection methods used, the formatting of the results, the desired accuracy of the comparison and other variables.

Satisfactory and Authentic

If the test results are within the predetermined range of the authentic or standard results, then the test preparation may be deemed satisfactory or authentic. As used herein, a determination of “satisfactory” or “authentic” signifies that the test preparation contains tracer and/or contains the tracer in a stated or suspected amount. For example, the methods can be used to determine whether a batch of soft drink, such as for example Coca-Cola®, is indeed the soft drink it claims to be, or whether it is different. The ability to do this is useful for product verification, particularly when a product is being produced at a number of different locations and using a number of different manufacturing processes and plants. Product verification is also useful for testing samples that are suspected of having degraded due to storage time or conditions. In this way, the shelf-life of a product can be determined.

It should be appreciated that an actual value of the detection need not be determined; a comparison of a test result to an authentic or standard result would allow the user to determine the satisfactory or authentic nature of the test sample.

### EXAMPLES

### Example 1

### Detection at PPB Levels of FD&C Dyes for Use as Authenticity Taggants Facilitated by the Use of Quaternary Ammonium Salts for Precipitation of Carmel Color

FD&C Blue #1 at 100 ppb

A caramel color containing beverage (75-100 ml) previously spiked with 100 ppb FD&C Blue #1 was dispensed into a plastic bottle; a cap was affixed and the sample was inverted and shaken to liberate any trapped carbonation. The cap was then loosened to allow carbonation to escape. This process was repeated 3-4 times until further inversion produces little or no carbonation. The beverage was slowly shaken either by hand or on a wrist action shaker for an additional 10-15 minutes with the cap loosened to allow the remaining carbonation to escape.

A 10 mL aliquot was dispensed into a 15 mL conical tube. A 250 μL sample of a cationic ammonium salt, (e.g. 3% myristyltrimethylammonium bromide) was added to the sample. KOH (25 μL, 50% solution) was added to sample to bring pH to ˜7.0. The sample was capped and gently shaken to precipitate the caramel color. The sample was placed on automatic vortexer for 10 minutes. The sample was then placed in centrifuge and centrifuged at 3000 rpm for 10 minutes. The supernatant was poured off into a fresh 15 mL conical tube. The precipitated pellet was discarded.

The sample (380 μL) was dispensed into 96-well microtiter plate. The sample was read by scanning from 300-750 nm at 1 nm increments in a Spectra MAX 250 spectrophotometer, available from Molecular Devices located in Sunnyvale, Calif. The detection of a peak at 610-660 nm showed adequate variation in absorbance verses FD&C Blue #1 concentration for identification of beverages containing FD&C Blue #1 from beverages that did not contain the dye taggant.

### Example 2

### Detection at PPB Levels of FD&C Dyes for Use as Authenticity Taggants Facilitated by the Use of Quaternary Ammonium Salts for Precipitation of Carmel Color

FD&C Blue #1 at 200 ppb

A caramel color containing beverage (75-100 ml) previously spiked with 200 ppb FD&C Blue #1 was dispensed into a plastic bottle; a cap was affixed and the sample was inverted and shaken to liberate trapped carbonation. The cap was then loosened to allow carbonation to escape. This process was repeated 3-4 times until further inversion produces little or no carbonation. The beverage was slowly shaken either by hand or on a wrist action shaker for an additional 10-15 minutes with the cap loosened to allow the remaining carbonation to escape.

A 10 mL aliquot was dispensed into a 15 mL conical tube. A 250 μL sample of a cationic ammonium salt, (e.g. 3% Ciba Zetag 7503) was added to the sample. KOH (25 μL, 50% solution) was added to the sample to bring pH to ˜7.0. The sample was capped and gently shaken to precipitate the caramel color. The sample was placed on automatic vortexer for 10 minutes. The sample was then placed in centrifuge and centrifuged at 3000 rpm for 10 minutes. Supernatant was poured off into a fresh 15 mL conical tube. The precipitated pellet was discarded.

The sample (380 μL) was dispensed into 96-well microtiter plate. The sample was read by scanning from 300-750 nm at 1 nm increments in the Spectra MAX 250 spectrophotometer. The detection of a peak at 610-660 nm showed adequate variation in absorbance verses FD&C Blue #1 concentration for identification of beverages containing FD&C Blue #1 from beverages that did not contain the dye taggant.

### Example 3

### Detection at PPB Levels of FD&C Dyes for Use as Authenticity Taggants Facilitated by the Use of Quaternary Ammonium Salts for Precipitation of Carmel Color

FD&C Red #40 at 500 ppb

A caramel color containing beverage (75-100 ml) previously spiked with 500 ppb FD&C Red #40 was dispensed into a plastic bottle; the cap was affixed and the sample was inverted and shaken to liberate trapped carbonation. The cap was then loosened to allow carbonation to escape. This process was repeated 3-4 times until further inversion produces little or no carbonation. The beverage was slowly shaken either by hand or on a wrist action shaker for an additional 10-15 minutes with the cap loosened to allow the remaining carbonation to escape.

A 10 mL aliquot was dispensed into a 15 mL conical tube. A 250 μL sample of a cationic ammonium salt, (e.g. 3% myristyltrimethylammonium bromide) was added to the sample. KOH (25 μL, 50% solution) was added to the sample to bring pH to ˜7.0. The sample was capped and gently shaken to precipitate the caramel color. The sample was placed on automatic vortexer for 10 minutes. The sample was then placed in centrifuge and centrifuged at 3000 rpm for 10 minutes. Supernatant was poured off into a fresh 15 mL conical tube. The precipitated pellet was discarded.

The sample (380 μL) was dispensed into 96-well microtiter plate. The sample was read by scanning from 300-750 nm at 1 nm increments in the Spectra MAX 250 spectrophotometer. The detection of a peak at 470-510 nm showed adequate variation in absorbance verses FD&C Red #40 concentration for identification of beverages containing FD&C Red #40 from beverages that did not contain the dye taggant.

### Example 4

### Detection at PPB Levels of FD&C Dyes for Use as Authenticity Taggants Facilitated by the Use of Ouaternary Ammonium Salts for Precipitation of Carmel Color

FD&C Yellow #5 at 2000 ppb

A caramel color containing beverage (75-100 ml) previously spiked with 2000 ppb FD&C Yellow #5 was dispensed into a plastic bottle; the cap was affixed and the sample was inverted and shaken to liberate trapped carbonation. The cap was then loosened to allow carbonation to escape. This process was repeated 3-4 times until further inversion produces little or no carbonation. The beverage was slowly shaken either by hand or on a wrist action shaker for an additional 10-15 minutes with the cap loosened to allow the remaining carbonation to escape.

A 10 mL aliquot was dispensed into a 15 mL conical tube. A 250 μL sample of a cationic ammonium salt, (e.g. 3% Ciba Zetag 7503) was added to the sample. KOH (25 μL, 50% solution) was added to the sample to bring pH to ˜7.0. The sample was capped and gently shaken to precipitate the caramel color. The sample was placed on automatic vortexer for 10 minutes. The sample was then placed in centrifuge and centrifuged at 3000 rpm for 10 minutes. Supernatant was poured off into a fresh 15 mL conical tube. The precipitated pellet was discarded.

The sample (380 μL) was dispensed into 96-well microtiter plate. The sample was read by scanning from 300-750 nm at 1 nm increments in the Spectra MAX 250 spectrophotometer. The detection of a peak at 400-440 nm showed adequate variation in absorbance verses FD&C Yellow #5 concentration for identification of beverages containing FD&C Yellow #5 from beverages that did not contain the dye taggant.

### Example 5

### Detection at PPB Levels of FD&C Dyes for Use as Authenticity Taggants Facilitated by the Use of Quaternary Ammonium Salts for Precipitation of Carmel Color

FD&C Blue #1 at: 0, 100, 200, 400, 1000 ppb

Caramel color containing beverage samples spiked with 0, 100, 200, 400, and 1000 ppb FD&C Blue #1 were dispensed into plastic bottles, caps were affixed, and samples (in turn) were inverted and shaken to liberate trapped carbonation. The caps were then loosened to allow carbonation to escape. The process was repeated 3-4 times until further inversion produces little or no carbonation. The beverages were slowly shaken on a wrist action shaker for 10-15 minutes with the caps loosened enough to allow the remaining carbonation to escape.

A 10 mL aliquot (for each sample) was dispensed into a 15 mL conical tube. A 250 μL sample of a cationic ammonium salt, (e.g. 3% Cetyl trimethyl ammonium Bromide) was added to each of the samples. KOH (25 μL, 50% solution) was added to each sample to bring pH to ˜7.0. The samples were capped and gently shaken to precipitate the caramel color. The samples were placed on automatic vortexer for 10 minutes. The samples were then placed in centrifuge and centrifuged at 3000 rpm for 10 minutes. The supernatant was poured off (for each sample) into fresh 15 mL conical tubes. The precipitated pellets for each sample were discarded.

The samples (380 μL) were dispensed into 96-well microtiter plate. The samples were read by scanning from 300-750 nm at 1 nm increments in the Spectra MAX 250 spectrophotometer. Detection of a peak at 610-660 nm (for each sample) showed adequate variation in absorbance verses FD&C Blue #1 concentration for identification of beverages containing FD&C Blue #1 from beverages that did not contain the dye taggant.

### Example 6

### Use of MYTAB Increases Percentage of Light Transmittance in a Treated Cola Beverage

| Index # | Sample | % Transmittance |
|---|---|---|
| 1 | Neat Pepsi | 1% |
| 2 | 5% Pepsi Soln | 67% |
| 3 | Pepsi Treated w/MYTAB | 54% |

This experiment illustrated that use of MYTAB treatment on cola samples increased transmittance of light through the sample over neat Pepsi.

“% Transmittance” is defined as the ratio of the initial intensity of a beam of light before it passes through a sample divided into the final intensity of the beam as seen by a detector after the beam passes through the sample. If I₀ is the initial intensity of the light beam and I is the final intensity of the light beam then % Transmittance is, by definition:

% Transmittance=I/I₀×100%

### Example 7

### Use of MYTAB Enables Detection of FD&C Blue #1 in a Cola Beverage

| | | Absorbance | Absorbance |
|---|---|---|---|
| | | Neat Cola | MYTAB |
| Index # | Sample | Beverage | Treated |
| 1 | Cola w/1000 ppb FD & C Blue | .37 | .08 |
| | 1 | | |
| 2 | Cola w/100 ppb FD & C Blue 1 | .37 | .03 |
| 3 | Cola w/0 ppb FD & C Blue 1 | .37 | .001 |

This experiment illustrated that the absorbance of a neat cola beverage did not change according to the concentration of FD&C Blue #1 present in the sample, but the absorbance did change linearly depending upon the concentration of FD&C Blue #1, when the sample was previously treated with MYTAB.

Absorbance is defined as:

Absorbance=−log₁₀(Transmittance)

### Example 8

### MYTAB Allows Detection of Sample containing 100 ppb FD&C Blue #1 Vs. Standard Cola Samples (Blinded Study)

| | Sample Treated with a Quater- | Absorbance @ | |
|---|---|---|---|
| Index # | nary Surfactant | 630 nm | Identified |
| 1 | Cola Sample 1 w/100 ppb FD & | .07 | X |
| | C Dye | | |
| 2 | Cola Sample 2 | .05 | |
| 3 | Cola Sample 3 w/100 ppb FD & | .07 | X |
| | C Dye | | |
| 4 | Cola Sample 4 | .05 | |
| 5 | Cola Sample 5 | .05 | |
| 6 | ola Sample 6 w/100 ppb FD & | .07 | X |
| | C Dye | | |
| 7 | Cola Sample 7 | .05 | |
| 8 | Cola Sample 8 w/100 ppb FD & | .07 | X |
| | C Dye | | |
| 9 | Cola Sample 9 | .05 | |
| 10 | Cola Sample 10 w/100 ppb | .07 | X |
| | FD & C Dye | | |

This experiment showed that a cola sample, which had previously been treated with a quaternary surfactant and contained 100 ppb FD&C Blue #1, consistently had a greater absorbance at 630 nm than an identical sample with no FD&C Blue #1 present.

For this blinded study, 5 unadulterated cola samples (e.g. index #s 2, 4, 5, 6 and 8) and 5 100 ppb FD&C Blue #1 spiked samples (e.g. index #s 1, 3, 6, 8 and 10) were analyzed without the operator knowing which samples were which. Once the samples were analyzed and termed as ‘spiked’ or ‘not spiked’, the actual contents of each sample was revealed. Absorbance was numerically higher in ‘spiked’ FD&C Blue #1 containing samples and these samples also exhibited a distinct peak centered at 630 nm that could be visually observed.

The foregoing written specification is to be considered to be sufficient to enable one skilled in the art to practice the invention. While the best mode for carrying out the invention has been described in detail, those skilled in the art to which this invention relates will recognize various alternative embodiments including those mentioned above as defined by the following claims. The examples disclosed herein are not to be construed as limiting of the invention as they are intended merely as illustrative of particular embodiments of the invention as enabled herein. Therefore, systems and methods that are functionally equivalent to those described herein are within the spirit and scope of the claims appended hereto. Indeed, various modifications of the invention in addition to those shown and described herein will become apparent to those skilled in the art from the foregoing description and fall within the scope of the appended claims.

All references, patents and patent publications that are recited in this application are incorporated in their entirety herein by reference.

We claim:

1. A method for detecting a tracer in a preparation, comprising:
contacting a sample of a preparation, wherein the preparation comprises a coloring agent and is suspected to comprise a tracer, with an effective amount of a precipitating agent to precipitate the coloring agent in the sample to yield a sample supernatant having a reduced concentration of coloring agent compared to the preparation; and
detecting the tracer in the sample supernatant.

2. The method of claim 1, wherein contacting the sample of the preparation comprises contacting the sample of the preparation having a coloring agent which interferes with detecting the tracer.

3. The method of claim 1, wherein contacting the sample of the preparation comprises contacting the sample of the preparation having a caramel coloring agent.

4. The method of claim 3, wherein contacting the sample of the preparation having the caramel coloring agent comprises contacting the sample of the preparation having a Type 1 caramel color.

5. The method of claim 4, wherein contacting the sample of the preparation with the effective amount of the precipitating agent to precipitate the Type 1 caramel color in the sample comprises contacting the sample of the preparation with the effective amount of at least one of a Type 2 caramel color and a Type 3 caramel color to precipitate the Type 1 caramel color in the sample.

6. The method of claim 1, wherein contacting the sample of the preparation with the effective amount of the precipitating agent comprises contacting the sample of the preparation with an effective amount of precipitating agent to precipitate the coloring agent and not the tracer.

7. The method of claim 1, wherein contacting the sample of the preparation with the effective amount of the precipitating agent comprises contacting the sample of the preparation with an effective amount of a cationic or quaternary surfactant or a polymer thereof.

8. The method of claim 1, wherein contacting the sample of the preparation with the effective amount of the precipitating agent comprises contacting the sample of the preparation with an effective amount of a quaternary ammonium bromide.

9. The method of claim 8, wherein contacting the sample of the preparation with the effective amount of the quaternary ammonium bromide comprises contacting the sample of the preparation with an effective amount of myristyltrimethylammonium bromide (MYTAB).

10. The method of claim 1, wherein contacting the sample of the preparation with the effective amount of the precipitating agent comprises contacting the sample of the preparation with an effective amount of a quaternary ammonium chloride.

11. The method of claim 1, wherein contacting the sample of the preparation with the effective amount of the precipitating agent comprises contacting the sample of the preparation with an effective amount of a copolymer of a quaternary acrylate salt and acylamide.

12. The method of claim 1, wherein contacting the sample of the preparation with the effective amount of the precipitating agent comprises contacting the sample of the preparation with an effective amount of a copolymer of sodium acrylate and acrylamide.

13. The method of claim 1, wherein contacting the sample of the preparation with the effective amount of the precipitating agent comprises contacting the sample of the preparation with an effective amount of at least one of a polyacrylamide and a polyamine.

14. The method of claim 1, wherein contacting the sample of the preparation comprises contacting a sample of a liquid consumable product.

15. The method of claim 14, wherein contacting the sample of the liquid consumable product comprises contacting a sample of a cola beverage.

16. The method of claim 1, wherein contacting the sample of the preparation with the effective amount of the precipitating agent to yield the sample supernatant having the reduced concentration of coloring agent comprises contacting the sample of the preparation with the effective amount of the precipitating agent to yield the sample supernatant having a reduced concentration between 0 and 10 percent.

17. The method of claim 1, wherein contacting the sample of the preparation with the effective amount of the precipitating agent to yield the sample supernatant having the reduced concentration of coloring agent comprises contacting the sample of the preparation with the effective amount of the precipitating agent to yield the sample supernatant having a reduced concentration between 0 and 1 percent.

18. The method of claim 1, wherein contacting the sample of the preparation with the effective amount of the precipitating agent to yield the sample. supernatant having the reduced concentration of coloring agent comprises contacting the sample of the preparation with the effective amount of the precipitating agent to yield the sample supernatant having a reduced concentration between 0 and 0.1 percent.

19. The method of claim 1, wherein detecting the tracer comprises directly detecting the tracer.

20. The method of claim 1, wherein detecting the tracer comprises indirectly detecting the tracer.

21. The method of claim 1, wherein contacting the sample of the preparation, wherein the preparation is suspected to comprise the tracer, comprises contacting the sample of the preparation, wherein the preparation is suspected to comprise a food dye.

22. The method of claim 21, wherein contacting the sample of the preparation, wherein the preparation is suspected to comprise the food dye, comprises contacting the sample of the preparation, wherein the preparation is suspected to comprise FD&C Blue #1.

23. The method of claim 1, wherein contacting the sample of the preparation, wherein the preparation is suspected to comprise the tracer, comprises contacting the sample of the preparation, wherein the preparation is suspected to comprise a light-sensitive compound.

24. The method of claim 1, wherein contacting the sample of the preparation, wherein the preparation is suspected to comprise the tracer, comprises contacting the sample of the preparation, wherein the preparation comprises the tracer in 100-2000 parts per billion (ppb) of the preparation.

25. The method of claim 1, wherein the detecting the tracer comprises detecting an optical property of the tracer.

26. The method of claim 25, wherein detecting the optical property of the tracer comprises detecting absorbance or emittance of the tracer at a predetermined wavelength.

27. The method of claim 1, wherein contacting the sample of the preparation, wherein the preparation comprises the coloring agent, with the effective amount of the precipitating agent to precipitate the coloring agent in the sample to yield the sample supernatant having the reduced concentration of coloring agent compared to the preparation comprises contacting a sample of a liquid consumable product, wherein the liquid consumable product comprises a caramel coloring agent, with an effective amount of MYTAB to precipitate the caramel coloring agent in the sample to yield a sample supernatant having a reduced concentration of caramel coloring agent compared to the liquid consumable product.

28. The method of claim 27, wherein contacting the sample of the liquid consumable product, wherein the liquid consumable product comprises the caramel coloring agent and is suspected to comprise the tracer, comprises contacting the sample of the liquid consumable product, wherein the liquid consumable product comprises the caramel coloring agent and is suspected to comprise a food dye.

29. The method of claim 27, wherein contacting the sample of the liquid consumable product with the effective amount of MYTAB to precipitate the caramel coloring agent in the sample further comprises raising the pH of the sample.

30. A method for verifying authenticity of a preparation, comprising:
contacting an authentic sample of an authentic preparation, wherein the authentic preparation comprises a tracer in an authentic amount and a coloring agent, with an authentic effective amount of a precipitating agent to precipitate the coloring agent in the authentic sample to yield an authentic sample supernatant having an authentic reduced concentration of coloring agent compared to the authentic preparation;
detecting the tracer in the authentic sample supernatant;
contacting a test sample of a test preparation, wherein the test preparation comprises the tracer in a test amount and the coloring agent, with a test effective amount of the precipitating agent to precipitate the coloring agent in the test sample to yield a test sample supernatant having a test reduced concentration of coloring agent compared to the test preparation;
detecting the tracer in the test sample supernatant; and
determining the test preparation is authentic when the tracer in the test sample supernatant is within a predetermined range of the tracer in the authentic sample supernatant.

31. The method of claim 30, wherein contacting the authentic sample of the authentic preparation comprises contacting the authentic sample of the authentic preparation having a coloring agent which interferes with detecting the tracer, and wherein contacting the test sample of the test preparation comprises contacting the test sample of the test preparation having the coloring agent which interferes with detecting the tracer.

32. The method of claim 30, wherein contacting the authentic sample of the authentic preparation comprises contacting the authentic sample of the authentic preparation having a caramel coloring agent, and wherein contacting the test sample of the test preparation comprises contacting the test sample of the test preparation having the caramel coloring agent.

33. The method of claim 32, wherein contacting the authentic sample of the authentic preparation having the caramel coloring agent comprises contacting the authentic sample of the authentic preparation having a Type 1 caramel color and wherein contacting the test sample of the test preparation having the caramel coloring agent comprises contacting the test sample of the test preparation having a Type 1 caramel color.

34. The method of claim 33, wherein contacting the authentic sample of the authentic preparation with the authentic effective amount of the precipitating agent to precipitate the Type 1 caramel color in the authentic sample comprises contacting the authentic sample of the authentic preparation with the authentic effective amount of at least one of a Type 2 caramel color and a Type 3 caramel color to precipitate the Type 1 caramel color in the authentic sample, and wherein contacting the test sample of the test preparation with the test effective amount of the precipitating agent to precipitate the Type 1 caramel color in the test sample comprises contacting the test sample of the test preparation with the test effective amount of at least one of a Type 2 caramel color and a Type 3 caramel color to precipitate the Type 1 caramel color in the test sample.

35. The method of claim 30, wherein contacting the authentic sample of the authentic preparation with the authentic effective amount of the precipitating agent comprises contacting the authentic sample of the authentic preparation with an authentic effective amount of the precipitating agent to precipitate the coloring agent and not the tracer, and wherein contacting the test sample of the test preparation with the test effective amount of the precipitating agent comprises contacting the test sample of the test preparation with a test effective amount of the precipitating agent to precipitate the coloring agent and not the tracer.

36. The method of claim 30, wherein contacting the authentic sample of the authentic preparation with the authentic effective amount of the precipitating agent comprises contacting the authentic sample of the authentic preparation with the authentic effective amount of a cationic or quaternary surfactant or a polymer thereof, and wherein contacting the test sample of the test preparation with the test effective amount of the precipitating agent comprises contacting the test sample of the test preparation with the test effective amount of the cationic or quaternary surfactant or the polymer thereof.

37. The method of claim 30, wherein contacting the authentic sample of the authentic preparation with the authentic effective amount of the precipitating agent comprises contacting the authentic sample of the authentic preparation with the authentic effective amount of a quaternary ammonium bromide, and wherein contacting the test sample of the test preparation with the test effective amount of the precipitating agent comprises contacting the test sample of the test preparation with the test effective amount of the quaternary ammonium bromide.

38. The method of claim 37, wherein contacting the authentic sample of the authentic preparation with the authentic effective amount of the quaternary ammonium bromide comprises contacting the authentic sample of the authentic preparation with the authentic effective amount of myristyltrimethylammonium bromide (MYTAB), and wherein contacting the test sample of the test preparation with the test effective amount of the quaternary ammonium bromide comprises contacting the test sample of the test preparation with the test effective amount of MYTAB.

39. The method of claim 30, wherein contacting the authentic sample of the authentic preparation with the authentic effective amount of the precipitating agent comprises contacting the authentic sample of the authentic preparation with the authentic effective amount of a quaternary ammonium chloride, and wherein contacting the test sample of the test preparation with the test effective amount of the precipitating agent comprises contacting the test sample of the test preparation with the test effective amount of the quaternary ammonium chloride.

40. The method of claim 30, wherein contacting the authentic sample of the authentic preparation with the authentic effective amount of the precipitating agent comprises contacting the authentic sample of the authentic preparation with the authentic effective amount of a copolymer of a quaternary acrylate salt and acylamide, and wherein contacting the test sample of the test preparation with the test effective amount of the precipitating agent comprises contacting the test sample of the test preparation with the test effective amount of the copolymer of the quaternary acrylate salt and acylamide.

41. The method of claim 30, wherein contacting the authentic sample of the authentic preparation with the authentic effective amount of the precipitating agent comprises contacting the authentic sample of the authentic preparation with the authentic effective amount of a copolymer of sodium acrylate and acrylamide, and wherein contacting the test sample of the test preparation with the test effective amount of the precipitating agent comprises contacting the test sample of the test preparation with the test effective amount of the copolymer of sodium acrylate and acrylamide.

42. The method of claim 30, wherein contacting the authentic sample of the authentic preparation with the authentic effective amount of the precipitating agent comprises contacting the authentic sample of the authentic preparation with the authentic effective amount of at least one of a polyacrylamide and a polyamine, and wherein contacting the test sample of the test preparation with the test effective amount of the precipitating agent comprises contacting the test sample of the test preparation with the test effective amount of the at least one of the polyacrylamide and the polyamine.

43. The method of claim 30, wherein contacting the authentic sample of the authentic preparation comprises contacting an authentic sample of an authentic liquid consumable product, and wherein contacting the test sample of the test preparation comprises contacting a test sample of a test liquid consumable product.

44. The combination of claim 43, wherein contacting the authentic sample of the authentic liquid consumable product comprises contacting an authentic sample of an authentic cola beverage, and wherein contacting the test sample of the test liquid consumable product comprises contacting a test sample of a test cola beverage.

45. The method of claim 30, wherein contacting the authentic sample of the authentic preparation with the authentic effective amount of the precipitating agent to yield the authentic sample supernatant having the authentic reduced concentration of coloring agent comprises contacting the authentic sample of the authentic preparation with the authentic effective amount of the precipitating agent to yield the authentic sample supernatant having an authentic reduced concentration between 0 and 10 percent, and wherein contacting the test sample of the test preparation with the test effective amount of the precipitating agent to yield the test sample supernatant having the test reduced concentration of coloring agent comprises contacting the test sample of the test preparation with the test effective amount of the precipitating agent to yield the test sample supernatant having a test reduced concentration between 0 and 10 percent.

46. The method of claim 30, wherein contacting the authentic sample of the authentic preparation with the authentic effective amount of the precipitating agent to yield the authentic sample supernatant having the authentic reduced concentration of coloring agent comprises contacting the authentic sample of the authentic preparation with the authentic effective amount of the precipitating agent to yield the authentic sample supernatant having an authentic reduced concentration between 0 and 1 percent, and wherein contacting the test sample of the test preparation with the test effective amount of the precipitating agent to yield the test sample supernatant having the test reduced concentration of coloring agent comprises contacting the test sample of the test preparation with the test effective amount of the precipitating agent to yield the test sample supernatant having a test reduced concentration between 0 and 1 percent.

47. The method of claim 30, wherein contacting the authentic sample of the authentic preparation with the authentic effective amount of the precipitating agent to yield the authentic sample supernatant having the authentic reduced concentration of coloring agent comprises contacting the authentic sample of the authentic preparation with the authentic effective amount of the precipitating agent to yield the authentic sample supernatant having an authentic reduced concentration between 0 and 0.1 percent, and wherein contacting the test sample of the test preparation with the test effective amount of the precipitating agent to yield the test sample supernatant having the test reduced concentration of coloring agent comprises contacting the test sample of the test preparation with the test effective amount of the precipitating agent to yield the test sample supernatant having a test reduced concentration between 0 and 0.1 percent.

48. The method of claim 30, wherein detecting the tracer in the authentic sample supernatant comprises directly detecting the tracer in the authentic sample supernatant, and wherein detecting the tracer in the test sample supernatant comprises directly detecting the tracer in the test sample supernatant.

49. The method of claim 30, wherein detecting the tracer in the authentic sample supernatant comprises indirectly detecting the tracer in the authentic sample supernatant, and wherein detecting the tracer in the test sample supernatant comprises indirectly detecting the tracer in the test sample supernatant.

50. The method of claim 30, wherein contacting the authentic sample of the authentic preparation, wherein the authentic preparation comprises the tracer, comprises contacting the authentic sample of the authentic preparation, wherein the authentic preparation comprises a food dye, and wherein contacting the test sample of the test preparation, wherein the test preparation comprises the tracer, comprises contacting the test sample of the test preparation, wherein the test preparation comprises the food dye.

51. The method of claim 50, wherein contacting the authentic sample of the authentic preparation, wherein the authentic preparation comprises the food dye, comprises contacting the authentic sample of the authentic preparation, wherein the authentic preparation comprises FD&C Blue #1, and wherein contacting the test sample of the test preparation, wherein the test preparation comprises the food dye, comprises contacting the test sample of the test preparation, wherein the test preparation comprises FD&C Blue #1.

52. The method of claim 30, wherein contacting the authentic sample of the authentic preparation, wherein the authentic preparation comprises the tracer, comprises contacting the authentic sample of the authentic preparation, wherein the authentic preparation comprises a light-sensitive compound, and wherein contacting the test sample of the test preparation, wherein the test preparation comprises the tracer, comprises contacting the test sample of the test preparation, wherein the test preparation comprises the light-sensitive compound.

53. The method of claim 30, wherein contacting the authentic sample of the authentic preparation, wherein the authentic preparation is suspected to comprise the authentic tracer, comprises contacting the authentic sample of the authentic preparation, wherein the authentic preparation comprises the tracer in 100-2000 parts per billion (ppb) of the authentic preparation, and wherein contacting the test sample of the test preparation, wherein the test preparation is suspected to comprise the test tracer, comprises contacting the test sample of the test preparation, wherein the test preparation comprises the tracer in 100-2000 ppb of the test preparation.

54. The method of claim 30, wherein detecting the tracer in the authentic sample supernatant comprises detecting an optical property of the tracer in the authentic sample supernatant, and wherein detecting the tracer in the test sample supernatant comprises detecting an optical property of the tracer in the test sample supernatant.

55. The method of claim 54, wherein detecting the optical property of the tracer in the authentic sample supernatant comprises detecting absorbance or emittance at a predetermined wavelength of the tracer in the authentic sample supernatant, and wherein detecting the optical property of the tracer in the test sample supernatant comprises detecting absorbance or emittance at a predetermined wavelength of the tracer in the test sample supernatant.

56. The method of claim 30, wherein contacting the authentic sample of the authentic preparation, wherein the authentic preparation comprises the coloring agent, with the authentic effective amount of the precipitating agent to precipitate the coloring agent in the authentic sample to yield the authentic sample supernatant having an authentic reduced concentration of coloring agent compared to the authentic preparation comprises contacting an authentic sample of an authentic liquid consumable product, wherein an authentic liquid consumable product comprises a caramel coloring agent, with an authentic effective amount of MYTAB to precipitate the caramel coloring agent in an authentic sample to yield an authentic sample supernatant having an authentic reduced concentration of caramel coloring agent compared to an authentic liquid consumable product, and wherein contacting the test sample of the test preparation, wherein the test preparation comprises the coloring agent, with the test effective amount of the precipitating agent to precipitate the coloring agent in the test sample to yield the test sample supernatant having the test reduced concentration of coloring agent compared to the test preparation comprises contacting a test sample of a test liquid consumable product, wherein a test liquid consumable product comprises a caramel coloring agent, with a test effective amount of MYTAB to precipitate the caramel coloring agent in a test sample to yield a test sample supernatant having a test reduced concentration of caramel coloring agent compared to the test liquid consumable product.

57. The method of claim 56, wherein contacting the authentic sample of the authentic liquid consumable product, wherein the authentic liquid consumable product comprises the tracer in an authentic amount, comprises contacting the authentic sample of the authentic liquid consumable product, wherein the authentic liquid consumable product comprises a food dye in the authentic amount and wherein contacting the test sample of the test liquid consumable product, wherein the test liquid consumable product comprises the tracer in the test amount, comprises contacting the test sample of the test liquid consumable product, wherein the test liquid consumable product comprises a food dye in a test amount.

58. The method of claim 56, wherein contacting the authentic sample of the authentic liquid consumable product with the authentic effective amount of MYTAB to precipitate the caramel coloring agent in the authentic sample further comprises raising the pH of the authentic sample, and wherein contacting the test sample of the test liquid consumable product with the test effective amount of MYTAB to precipitate the caramel coloring agent in the test sample further comprises raising the pH of the test sample.

59. A method of quality validation of a preparation, comprising:
contacting a standard sample of a standard preparation, wherein the standard preparation comprises a tracer in a standard amount and a coloring agent, with a standard effective amount of a precipitating agent to precipitate the coloring agent in the standard sample to yield a standard sample supernatant having a standard reduced concentration of coloring agent compared to the standard preparation;
detecting the tracer in the standard sample supernatant to produce a standard result;
contacting a test sample of a test preparation, wherein the test preparation comprises the tracer in a test amount and the coloring agent, with a test effective amount of the precipitating agent to precipitate the coloring agent in the test sample to yield a test sample supernatant having a test reduced concentration of coloring agent compared to the test preparation;
detecting the tracer in the test sample supernatant to produce a test result; and
determining the test preparation is satisfactory when the test result is within a predetermined range of the standard result.

60. The method of claim 59, wherein contacting the standard sample of the standard preparation comprises contacting a standard sample of the standard preparation having a coloring agent which interferes with detecting the tracer, and wherein contacting the test sample of the test preparation comprises contacting the test sample of the test preparation having the coloring agent which interferes with detecting the tracer.

61. The method of claim 59, wherein contacting the standard sample of the standard preparation comprises contacting the standard sample of the standard preparation having a caramel coloring agent, and wherein contacting the test sample of the test preparation comprises contacting the test sample of the test preparation having the caramel coloring agent.

62. The method of claim 61, wherein contacting a standard sample of the standard preparation having the caramel coloring agent comprises contacting a standard sample of a standard preparation having a Type 1 caramel color, and wherein contacting the test sample of the test preparation having the caramel coloring agent comprises contacting the test sample of the test preparation having a Type 1 caramel color.

63. The method of claim 62, wherein contacting the standard sample of the standard preparation with the standard effective amount of the precipitating agent to precipitate the Type 1 caramel color in the standard sample comprises contacting the standard sample of the standard preparation with the standard effective amount of at least one of a Type 2 caramel color and a Type 3 caramel color to precipitate the Type 1 caramel color in the standard sample, and wherein contacting the test sample of the test preparation with the test effective amount of the precipitating agent to precipitate the Type 1 caramel color in the test sample comprises contacting the test sample of the test preparation with the test effective amount of at least one of a Type 2 caramel color and a Type 3 caramel color to precipitate the Type 1 caramel color in the test sample.

64. The method of claim 59, wherein contacting the standard sample of the standard preparation with the standard effective amount of the precipitating agent comprises contacting the standard sample of the standard preparation with a standard effective amount of the precipitating agent to precipitate the coloring agent and not the tracer, and wherein contacting the test sample of the test preparation with the test effective amount of the precipitating agent comprises contacting the test sample of the test preparation with a test effective amount of the precipitating agent to precipitate the coloring agent and not the tracer.

65. The method of claim 59, wherein contacting the standard sample of the standard preparation with the standard effective amount of the precipitating agent comprises contacting the standard sample of the standard preparation with the standard effective amount of a cationic or quaternary surfactant or a polymer thereof, and wherein contacting the test sample of the test preparation with the test effective amount of the precipitating agent comprises contacting the test sample of the test preparation with the test effective amount of the cationic or quaternary surfactant or the polymer thereof.

66. The method of claim 59, wherein contacting the standard sample of the standard preparation with the standard effective amount of the precipitating agent comprises contacting the standard sample of the standard preparation with the standard effective amount of a quaternary ammonium bromide, and wherein contacting the test sample of the test preparation with the test effective amount of the precipitating agent comprises contacting the test sample of the test preparation with the test effective amount of the quaternary ammonium bromide.

67. The method of claim 66, wherein contacting the standard sample of the standard preparation with the standard effective amount of the quaternary ammonium bromide comprises contacting the standard sample of the standard preparation with the standard effective amount of myristyltrimethylammonium bromide (MYTAB), and wherein contacting the test sample of the test preparation with the test effective amount of the quaternary ammonium bromide comprises contacting the test sample of the test preparation with the test effective amount of MYTAB.

68. The method of claim 59, wherein contacting the standard sample of the standard preparation with the standard effective amount of the precipitating agent comprises contacting the standard sample of the standard preparation with the standard effective amount of a quaternary ammonium chloride, and wherein contacting the test sample of the test preparation with the test effective amount of the precipitating agent comprises contacting the test sample of the test preparation with the test effective amount of the quaternary ammonium chloride.

69. The method of claim 59, wherein contacting the standard sample of the standard preparation with the standard effective amount of the precipitating agent comprises contacting the standard sample of the standard preparation with the standard effective amount of a copolymer of a quaternary acrylate salt and acylamide, and wherein contacting the test sample of the test preparation with the test effective amount of the precipitating agent comprises contacting the test sample of the test preparation with the test effective amount of the copolymer of the quaternary acrylate salt and acylamide.

70. The method of claim 59, wherein contacting the standard sample of the standard preparation with the standard effective amount of the precipitating agent comprises contacting the standard sample of the standard preparation with the standard effective amount of a copolymer of sodium acrylate and acrylamide, and wherein contacting the test sample of the test preparation with the test effective amount of the precipitating agent comprises contacting the test sample of the test preparation with the test effective amount of the copolymer of sodium acrylate and acrylamide.

71. The method of claim 59, wherein contacting the standard sample of the standard preparation with the standard effective amount of the precipitating agent comprises contacting the standard sample of the standard preparation with the standard effective amount of at least one of a polyacrylamide and a polyamine, and wherein contacting the test sample of the test preparation with the test effective amount of the precipitating agent comprises contacting the test sample of the test preparation with the test effective amount of the at least one of the polyacrylamide and the polyamine.

72. The method of claim 59, wherein contacting the standard sample of the standard preparation comprises contacting a standard sample of a standard liquid consumable product, and wherein contacting the test sample of the test preparation comprises contacting a test sample of a test liquid consumable product.

73. The combination of claim 72, wherein contacting the standard sample of the standard liquid consumable product comprises contacting a standard sample of a standard cola beverage, and wherein contacting the test sample of the test liquid consumable product comprises contacting a test sample of a test cola beverage.

74. The method of claim 59, wherein contacting the standard sample of the standard preparation with the standard effective amount of the precipitating agent to yield the standard sample supernatant having the standard reduced concentration of coloring agent comprises contacting the standard sample of the standard preparation with the standard effective amount of the precipitating agent to yield the standard sample supernatant having a standard reduced concentration between 0 and 10 percent, and wherein contacting the test sample of the test preparation with the test effective amount of the precipitating agent to yield the test sample supernatant having the test reduced concentration of coloring agent comprises contacting the test sample of the test preparation with the test effective amount of the precipitating agent to yield the test sample supernatant having a test reduced concentration between 0 and 10 percent.

75. The method of claim 59, wherein contacting the standard sample of the standard preparation with the standard effective amount of the precipitating agent to yield the standard sample supernatant having the standard reduced concentration of coloring agent comprises contacting the standard sample of the standard preparation with the standard effective amount of the precipitating agent to yield the standard sample supernatant having a standard reduced concentration between 0 and 1 percent, and wherein or contacting the test sample of the test preparation with the test effective amount of the precipitating agent to yield the test sample supernatant having the test reduced concentration of coloring agent comprises contacting the test sample of the test preparation with the test effective amount of the precipitating agent to yield the test sample supernatant having a test reduced concentration between 0 and 1 percent.

76. The method of claim 59, wherein contacting the standard sample of the standard preparation with the standard effective amount of the precipitating agent to yield the standard sample supernatant having the standard reduced concentration of coloring agent comprises contacting the standard sample of the standard preparation with the standard effective amount of the precipitating agent to yield the standard sample supernatant having a standard reduced concentration between 0 and 0.1 percent, and wherein contacting the test sample of the test preparation with the test effective amount of the precipitating agent to yield the test sample supernatant having the test reduced concentration of coloring agent comprises contacting the test sample of the test preparation with the test effective amount of the precipitating agent to yield the test sample supernatant having a test reduced concentration between 0 and 0.1 percent.

77. The method of claim 59, wherein detecting the tracer in the standard sample supernatant comprises directly detecting the tracer in the standard sample supernatant, and wherein detecting the tracer in the test sample supernatant comprises directly detecting the tracer in the test sample supernatant.

78. The method of claim 59, wherein detecting the tracer in the standard sample supernatant comprises indirectly detecting the tracer in the standard sample supernatant, and wherein detecting the tracer in the test sample supernatant comprises indirectly detecting the tracer in the test sample supernatant.

79. The method of claim 59, wherein contacting the standard sample of the standard preparation, wherein the standard preparation comprises the tracer, comprises contacting the standard sample of the standard preparation, wherein the standard preparation comprises a food dye, and wherein contacting the test sample of the test preparation, wherein the test preparation comprises the tracer, comprises contacting the test sample of the test preparation, wherein the test preparation comprises the food dye.

80. The method of claim 79, wherein contacting the standard sample of the standard preparation, wherein the standard preparation comprises the food dye, comprises contacting the standard sample of the standard preparation, wherein the standard preparation comprises FD&C Blue #1, and wherein contacting the test sample of the test preparation, wherein the test preparation comprises the food dye, comprises contacting the test sample of the test preparation, wherein the test preparation comprises FD&C Blue #1.

81. The method of claim 59, wherein contacting the standard sample of the standard preparation, wherein the standard preparation comprises the tracer, comprises contacting the standard sample of the standard preparation, wherein the standard preparation comprises a light-sensitive compound, and wherein contacting the test sample of the test preparation, wherein the test preparation comprises the tracer, comprises contacting the test sample of the test preparation, wherein the test preparation comprises the light-sensitive compound.

82. The method of claim 59, wherein contacting the standard sample of the standard preparation, wherein the standard preparation is suspected to comprise the standard tracer, comprises contacting the standard sample of the standard preparation, wherein the standard preparation comprises the tracer in 100-2000 parts per billion (ppb) of the standard preparation, and wherein contacting the test sample of the test preparation, wherein the test preparation is suspected to comprise the test tracer, comprises contacting the test sample of the test preparation, wherein the test preparation comprises the tracer in 100-2000 ppb of the test preparation.

83. The method of claim 59, wherein detecting the tracer in the standard sample supernatant comprises detecting an optical property of the tracer in the standard sample supernatant, and wherein detecting the tracer in the test sample supernatant comprises detecting an optical property of the tracer in the test sample supernatant.

84. The method of claim 83, wherein detecting the optical property of the tracer in the standard sample supernatant comprises detecting absorbance or emittance at a predetermined wavelength of the tracer in the standard sample supernatant, and wherein detecting the optical property of the tracer in the test sample supernatant comprises detecting absorbance or emittance at a predetermined wavelength of the tracer in the test sample supernatant.

85. The method of claim 59, wherein contacting the standard sample of the standard preparation, wherein the standard preparation comprises the coloring agent, with the standard effective amount of the precipitating agent to precipitate the coloring agent in the standard sample to yield the standard sample supernatant having a standard reduced concentration of coloring agent compared to the standard preparation comprises contacting the standard sample of a standard liquid consumable product, wherein a standard liquid consumable product comprises a caramel coloring agent, with a standard effective amount of MYTAB to precipitate the caramel coloring agent in a standard sample to yield a standard sample supernatant having a standard reduced concentration of caramel coloring agent compared to the standard liquid consumable product and wherein contacting the test sample of a test preparation, wherein the test preparation comprises the coloring agent, with the test effective amount of the precipitating agent to precipitate the coloring agent in the test sample to yield the test sample supernatant having the test reduced concentration of coloring agent compared to the test preparation comprises contacting a test sample of a test liquid consumable product, wherein a test liquid consumable product comprises a caramel coloring agent, with a test effective amount of MYTAB to precipitate the caramel coloring agent in a test sample to yield the test sample supernatant having the test reduced concentration of caramel coloring agent compared to the test liquid consumable product.

86. The method of claim 85, wherein contacting the standard sample of the standard liquid consumable product, wherein the standard liquid consumable product comprises the tracer in the standard amount, comprises contacting the standard sample of the standard liquid consumable product, wherein the standard liquid consumable product comprises a food dye in the standard amount, and wherein contacting the test sample of the test liquid consumable product, wherein the test liquid consumable product comprises the tracer in the test amount, comprises contacting the test sample of the test liquid consumable product, wherein the test liquid consumable product comprises a food dye in the test amount.

87. The method of claim 85, wherein contacting the standard sample of the standard liquid consumable product with the standard effective amount of MYTAB to precipitate the caramel coloring agent in the standard sample further comprises raising the pH of the standard sample, and wherein contacting the test sample of the test liquid consumable product with the test effective amount of MYTAB to precipitate the caramel coloring agent in the test sample further comprises raising the pH of the test sample.

88. A method of analyzing a product, the product comprising a coloring agent and suspected of comprising a tracer, the method comprising:
contacting at least a sample of the product with an effective amount of a precipitating agent to precipitate the coloring agent in the sample to yield a supernatant of the product having a reduced concentration of coloring agent compared to the sample of the product; and
detecting the tracer in the supernatant of the product.

89. The method of claim 88, further comprising preparing a liquid preparation of at least a portion of the product if the product is in a non-liquid form, and wherein contacting the at least the sample of the product with the effective amount of the precipitating agent to precipitate the coloring agent in the sample to yield the supernatant having the reduced concentration of coloring agent compared to the sample comprises contacting the at least the sample of the liquid preparation of the product with the effective amount of the precipitating agent to precipitate the coloring agent in the sample of the liquid preparation to yield the supernatant of the liquid preparation having the reduced concentration of coloring agent compared to the sample of the liquid preparation, and wherein detecting the tracer in the supernatant of the product comprises detecting the tracer in the supernatant of the liquid preparation of the product.

90. The method of claim 88, wherein contacting the at least the sample of the product with the effective amount of the precipitating agent to precipitate the coloring agent in the sample to yield the supernatant of the product having the reduced concentration of coloring agent compared to the sample of the product comprises contacting the product with the effective amount of the precipitating agent to precipitate the coloring agent in the product to yield the supernatant of the product having the reduced concentration of coloring agent compared to the product.

91. The method of claim 88, wherein detecting the tracer in the supernatant of the product comprises detecting a presence of the tracer in the supernatant of the product.

92. The method of claim 88, wherein detecting the tracer in the supernatant of the product comprises detecting a concentration of the tracer in the supernatant of the product.
